(19) United States
(12) Patent Application Publication
(10) Pub. No.: US 2010/0084578 A1
(43) Pub. Date: Apr. 8, 2010

(54) ELECTRON BEAM LITHOGRAPHY APPARATUS AND STAGE MECHANISM THEREOF

(75) Inventor: Hiroaki Kitahara, Higashi-Matsuyama (JP)

Correspondence Address: SUGHRUE MION, PLLC, 2100 PENNSYLVANIA AVENUE, N.W., SUITE 800, WASHINGTON, DC, 20037, US

(73) Assignee: Pioneer Corporation, Meguro-ku, Tokyo (JP)

(21) Appl. No.: 12/593,259

(22) PCT Filed: Mar. 28, 2007
(86) PCT No.: PCT/JP2007/056705

§ 371 Date: Sep. 25, 2009

(51) Int. Cl. A61N 5/00

(52) U.S. Cl. 250/492.3

(57) ABSTRACT

A stage mechanism which comprises a positioning mechanism including a rotating stage and a linear movement stage, the positioning mechanism being housed in a vacuum chamber, and pipes leading from the outside of the vacuum chamber to the positioning mechanism. In the vacuum chamber, a trench-like space spreading underneath the positioning mechanism is provided, and the pipes connected between the positioning mechanism and vacuum bulkheads are laid to be shaped substantially like a U so as to deform in response to movement of the linear movement stage without touching an inside wall or the like of the vacuum chamber in this space.

### TECHNICAL FIELD

The present invention relates to an electron beam lithography apparatus and a stage mechanism thereof that can be used in a mastering process for a record disc master or the like.

### BACKGROUND ART

In the mastering process for a master of optical discs, pits and grooves are formed usually by exposing a resist film to a laser beam. A laser beam recorder (LBR) used in this mastering process uses as its light source UV (ultraviolet), Deep-UV (far ultraviolet) with which the beam diameter can be further reduced, or the like. The exposure limit of a laser beam, that is, the record density of an optical disc is determined by the light diffraction limit determined by the light wavelength and the numerical aperture of an object lens. In recent years, an electron beam lithography method used in a semiconductor process has been investigated for the purpose of forming minute pits or grooves sized beyond the exposure limit of a laser beam to improve the record density of optical discs. By adopting this electron beam lithography method, the master production for discrete track media, patterned media, and the like that are promising as future technology for high density hard disks as well as for high density optical discs such as blue ray discs is expected to be dealt with.

FIG. 1 shows the configuration of a rotating stage-type electron beam lithography apparatus used in mastering disc masters. The rotating stage-type electron beam lithography apparatus comprises an X-stage 100, a rotating stage 101 mounted on the X-stage 100, and an electronic optical lens barrel 103. A disc master 200 is mounted on a turn table 104 on the rotating stage, and at the same time that the rotating stage 101 is driven to rotate, the X-stage 100 is moved in a disc radial direction (in a direction of arrow A in FIG. 1), thereby performing the positioning control of the electron beam irradiation position. Since being constituted by a combination of a linear movement stage (the X-stage) and the rotating stage (a θ-stage) as above, the stage mechanism of the electron beam lithography apparatus is usually called an X-θ stage. Because positioning accuracy on the nanoscale is required of the rotating stage used in electron beam lithography apparatuses, an air spindle is usually used in the rotating stage. Meanwhile, since the electron beam has a characteristic of being considerably diffused and attenuated in the atmosphere, the irradiation path for the electron beam needs to be evacuated, and thus the entire stage is placed in a vacuum chamber 105. Hence, conventionally, an air spindle contained in a sealed vacuum container with the rotation shaft to which a shaft seal for vacuum using magnetic fluid, differential exhaust, or the like, is applied has been used as the rotating stage. Further, in order to remove vibrations occurring in the apparatus, the entire apparatus is mounted on a vibration-free deck 106.

In order to make the air spindle operate in the vacuum chamber, measures of some kind are needed which introduces high pressure air for an air bearing, electric signals for motor drive, and the like from outside of the vacuum chamber into the air spindle and which exhausts air used in the bearing out of the vacuum chamber. As this means, conventionally, a flexible pipe such as a bellows or a flexible tube has been used. Here, since the rotating stage is mounted on the X-stage, the pipe connecting to the air spindle deforms in response to the movement of the X-stage. The deformation of the pipe during the movement of the X-stage may hinder the feed operation of the X-stage, thus preventing highly accurate positioning control. Jpn. Appl. Phys. Vol. 40 (2001) PP. 1653-1660 (Non-patent reference 1) discloses a piping method where a metallic flexible tube 107 leading from the side of the rotating stage upper portion to a bulkhead of the vacuum chamber is laid to be shaped like a “U” turned on its side as shown in FIG. 2. In this case, the flexible tube 107 responds to the stroke of the X-stage with the deformation as shown in FIG. 3. In the case of this configuration, a support member to support the weight of the flexible tube is needed, and the bottom of the vacuum chamber or the like actually takes on that role. When viewing the actual movement microscopically, as the tube moves deforming, friction occurs between the support member (the bottom of the vacuum chamber or the like) and the flexible tube, which causes frictional resistance and minute vibrations, resulting in a decrease in the movement accuracy of the X-stage. Further, since a high pressure air supplying tube for the bearing, motor driving feeder lines, and the like are together passed through its inside, a flexible tube of a relatively large diameter (an inch or greater in inner diameter) has been used conventionally. Thus, the load on the X-stage due to the flexural stiffness thereof has been a cause of a decrease in the movement accuracy of the X-stage. If a differential exhaust seal that is fully noncontact is adopted to improve the rotation accuracy of the spindle motor, the number of tubes is further increased, which makes the above problem more serious.

Meanwhile, Jpn. Appl. Phys. Vol. 43 (2004) PP. 5068-5073 (Non-patent reference 2) and Japanese Patent Application Laid-Open Publication No. 2003-287146 (Reference 1) disclose an apparatus equipped with a bellows elastic in the X-direction. However, the spring force of the bellows and the sliding frictional resistance of a guiding mechanism provided to prevent the bellows from buckling cause a decrease in the movement accuracy of the X-stage.

### Disclosure of the Invention

### Technical Problems

The present invention was made in view of the above problem, and an object thereof is to provide a stage mechanism capable of more highly accurate positioning control by reducing the movement load on the X-stage due to the rotating stage piping, and an electron beam lithography apparatus equipped with this stage mechanism.

### Solution for Solving the Problems

According to the present invention, there is provided a stage mechanism which comprises a positioning mechanism including a rotating stage having a turn table, and a linear movement stage to linearly move the rotating stage for positioning; a vacuum chamber housing the positioning mechanism; and at least one flexible pipe to make the outside of the vacuum chamber and the inside of the positioning mechanism communicate. The vacuum chamber has a lower space that spreads underneath the underside of the positioning mechanism, and the pipe leads from a surface of the positioning mechanism through the lower space to an inside wall of the vacuum chamber.

According to the present invention, there is provided an electron beam lithography apparatus which comprises a positioning mechanism including a rotating stage having a turn table, and a linear movement stage to linearly move the rotating stage for positioning; an electron beam irradiating means to irradiate an electron beam onto a disc master mounted on the rotating stage to form record marks; a vacuum chamber housing the positioning mechanism; and at least one flexible pipe to make the outside of the vacuum chamber and the inside of the positioning mechanism communicate. The vacuum chamber has a lower space that spreads underneath the underside of the positioning mechanism, and the pipe leads from a surface of the positioning mechanism through the lower space to an inside wall of the vacuum chamber.

### BRIEF DESCRIPTION OF DRAWINGS

FIG. 1 shows the configuration of a conventional electron beam lithography apparatus;

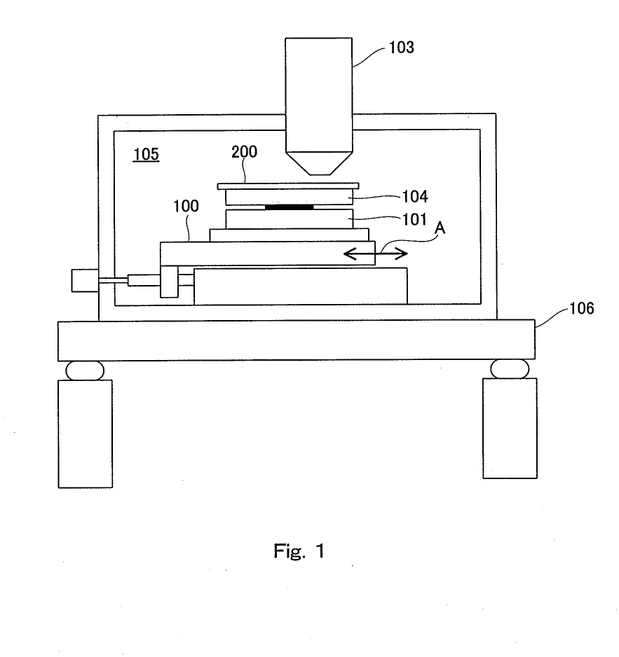

FIG. 2 shows a piping method in the conventional electron beam lithography apparatus;

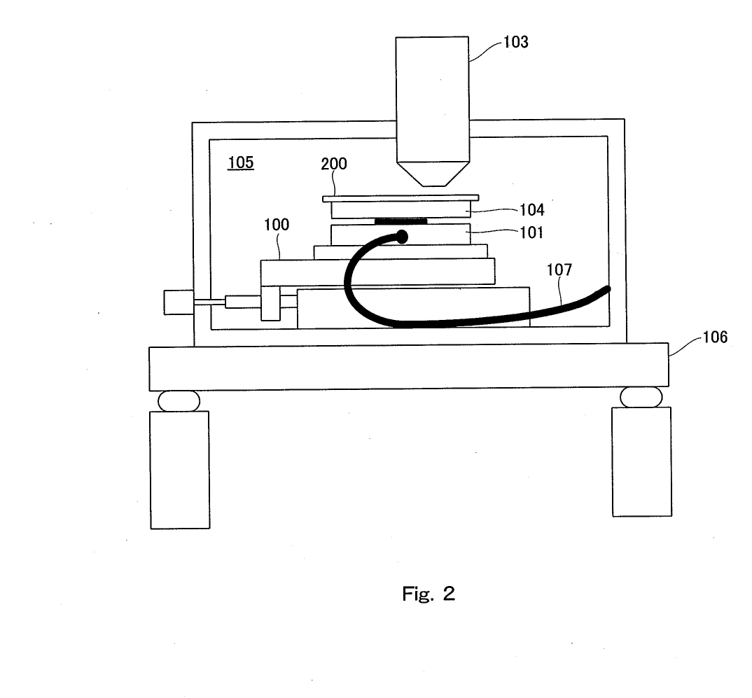

FIG. 3 shows the movement of a pipe of the conventional electron beam lithography apparatus;

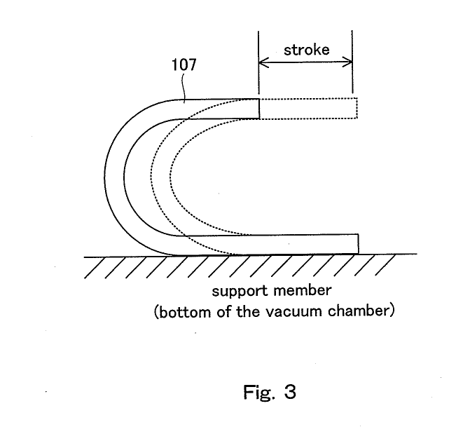

FIG. 4 shows the configuration of a rotating stage in an embodiment of the present invention;

FIG. 5 shows the configuration of an electron beam lithography apparatus that is an embodiment of the present invention;

FIG. 6 shows the movement of a pipe of the electron beam lithography apparatus that is an embodiment of the present invention;

FIG. 7 shows the configuration of an electron beam lithography apparatus that is an embodiment of the present invention; and

FIG. 8 shows the configuration of an electron beam lithography apparatus that is another embodiment of the present invention.

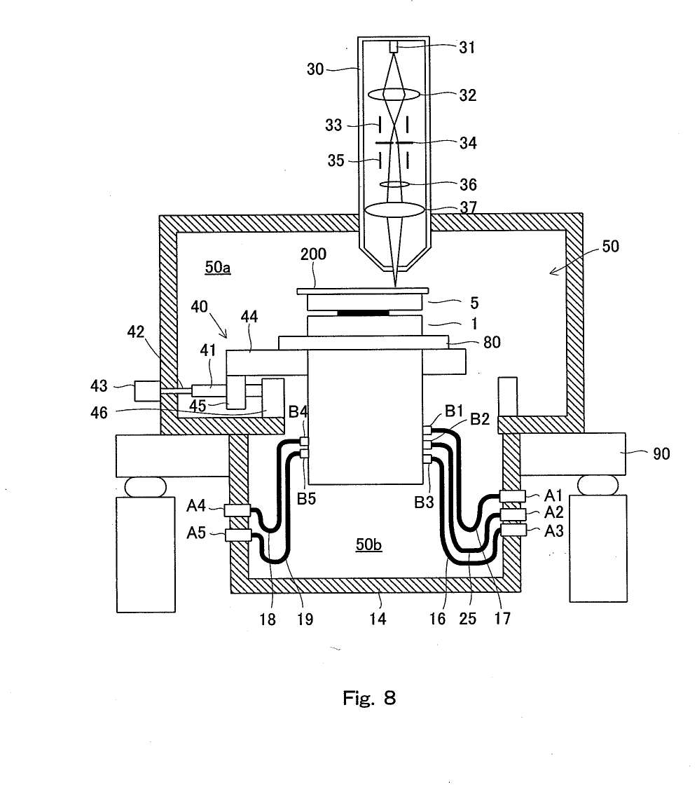

### EXPLANATION OF REFERENCE NUMERALS

1 Rotating stage
5 Turn table
13 Housing
14 Vacuum bulkhead
16-19 Flexible tube
25 Feeder line
30 Electronic optical lens barrel
40 X-stage
50 Vacuum chamber
50a Upper region (Upper space)
50b Lower region (Lower space)
A1-A5 Feedthrough
B1-B5 Feedthrough

### DETAILED DESCRIPTION OF THE INVENTION

Embodiments of the present invention will be described with reference to the drawings. The same reference numerals are used to denote substantially the same or equivalent constituents or parts throughout the figures cited below.

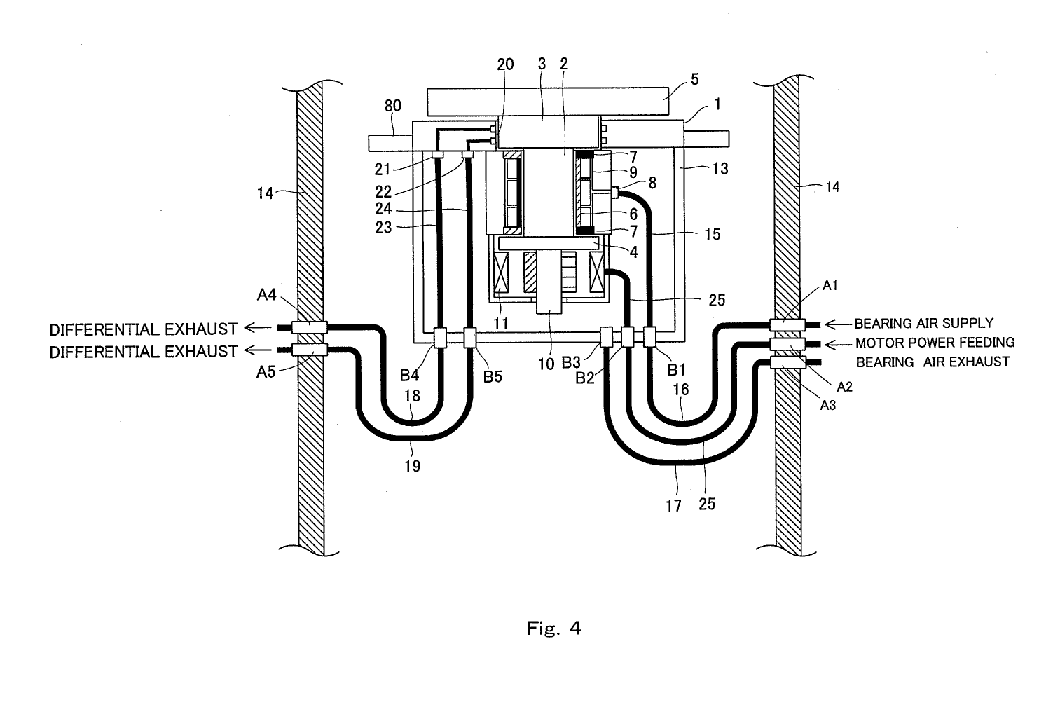

FIG. 4 is a cross-sectional view of a rotating stage 1 using an air spindle that is an embodiment of the present invention. Thrust plates 3, 4 and a turn table 5 are connected to a rotation shaft 2 of the rotating stage 1, and these form a rotating section. A journal bearing 6 and thrust bearings 7 are placed to face the rotation shaft 2 and the thrust plates 3, 4 via minute spaces respectively. When compressed air is supplied into a bearing inlet 8, the compressed air comes out of nozzles forming parts of the journal bearing 5 and thrust bearings 6 via a supply passageway 9. The rotation shaft 2 and the thrust plates 3, 4 are supported by this compressed air in a noncontact way. Under the thrust plate 4, there are provided a shaft 10 linked to the rotation shaft 2 and an AC servo motor 11 supplying driving force to the shaft 10 in a noncontact way. Air leakage from the rotation shaft is prevented through sealing by differentially exhausting a plurality of noncontact seal portions 20 provided adjacent the shaft. As such, the rotating section of the rotating stage 1 is supported in a completely noncontact way with respect to the fixed section, and hence frictional resistance associated with rotation is suppressed to be small, realizing ultra-highly accurate rotation control.

Of the above constituents of the air spindle, the ones other than the turn table 5 are housed in an airtight way in a housing 13. Vacuum bulkheads 14 are walls separating the inside and outside of a vacuum chamber in an airtight way, and the rotating stage 1 is placed inward of the vacuum bulkheads 14, that is, in the vacuum atmosphere inside the vacuum chamber. Note that there is atmospheric pressure outward of the vacuum bulkheads 14 and inside the housing 13.

Feedthroughs A1 to A5 are provided on the vacuum bulkheads 14, and feedthroughs B1 to B5 are provided on the bottom of the housing 13 of the rotating stage 1. The feedthrough forms a linkage of pipes or wires inward and outward of a bulkhead, and its airtightness is secured so that air does not leak into the vacuum chamber through the linkage. Piping and wiring over inward and outward of the vacuum bulkheads 14 are implemented via these feedthroughs. Namely, the supply of compressed air to the air spindle, the exhaust of air from the air spindle, and the supply of drive electric power to the AC servo motor 11 are implemented via the feedthroughs. An air supply pipe 15 is connected between the bearing inlet 8 and the feedthrough B1. A small-diameter flexible tube 16 of, e.g., ½ inch or less in outer diameter that is made of metal such as stainless is connected between the feedthrough B1 and the feedthrough A1. By this piping, compressed air can be supplied to the air spindle from outward of the vacuum bulkheads 14 with maintaining the degree of vacuum in the vacuum chamber. Further, feeder lines 25 are connected respectively between the AC servo motor 11 and the feedthrough B2 and between the feedthrough B2 and the feedthrough A2. By this means, drive electric power is supplied to the AC servo motor 11 from outward of the vacuum bulkheads 14 with maintaining the degree of vacuum in the vacuum chamber. Moreover, a small-diameter flexible tube 17 of, e.g., ½ inch or less is connected between the feedthrough B3 and the feedthrough A3. By this means, part of the compressed air supplied to the vacuum bearings 5, 6 is exhausted outward of the vacuum bulkheads 14 via the flexible tube 17. Yet further, exhaust pipes 23, 24 are connected respectively between bearing outlets 21, 22 and the feedthrough B4, B5, and small-diameter flexible tubes 18, 19 of, e.g., ½ inch or less are connected respectively between the feedthrough B4, B5 and the feedthrough A4, A5. By this piping, air which has flowed into the noncontact seal portions 20 can be differentially exhausted outward of the vacuum bulkheads 14.

Each of the flexible tubes 16 to 19 and the feeder line 25 connecting the feedthroughs B1 to B5 provided on the bottom of the housing 13 and the feedthroughs A1 to A5 provided on the vacuum bulkheads 14, is laid hanging down in the middle part to be shaped substantially like a “U” as shown in FIG. 4. As such, in the stage mechanism according to the present invention, the pipes and wires for air supply to and exhaust from the air spindle and for power feeding thereto are individually provided without the use of parts such as bellows or guiding tubes used in conventional apparatuses.

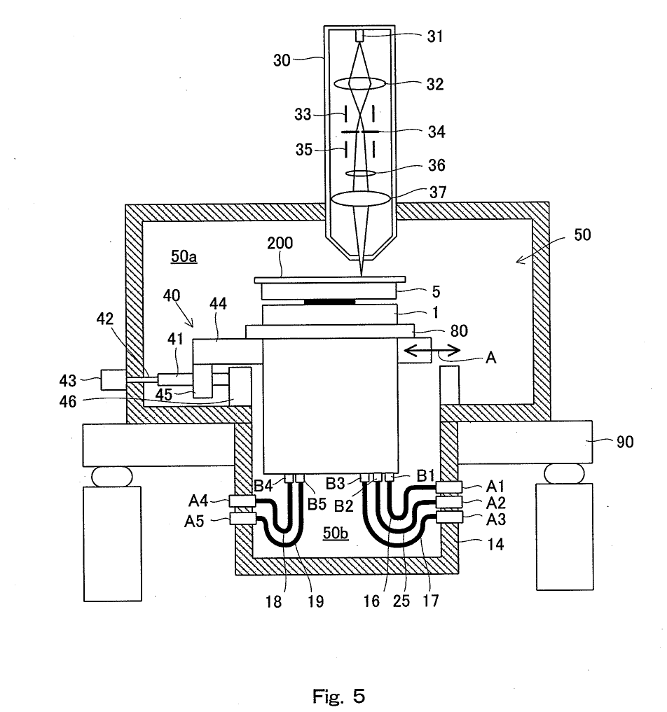

FIG. 5 shows the configuration of an electron beam lithography apparatus provided with the above rotating stage 1 according to the present invention. An electronic optical lens barrel 30 comprises an electron gun 31, a condenser lens 32, a blanking electrode 33, an aperture 34, a deflector 35, a focus lens 36, an object lens 37, and the like. Electrons emitted from the electron gun 31 are converged to the center of the blanking electrode by the condenser lens 32 to make a cross-over point, then pass through the aperture 34 and the deflector 35, and are converged onto a record master by the object lens 37. The electron beam is modulated by deflecting the electron beam with the blanking electrode and blocking it with the aperture.

A vacuum chamber 50 housing an X-stage 40 and the rotating stage 1 in an airtight way is provided with a trench-like space that spreads underneath the bottom of the X-stage 40. The upper portion of the rotating stage including the X-stage 40 and the turn table 5 is housed in a space 50a (hereinafter called a first region) above the trench-like space. Meanwhile, the lower portion of the rotating stage and the pipes for air supply to and exhaust from the air spindle and for power feeding thereto are placed in the trench-like space 50b (hereinafter called a second region). As shown in FIG. 5, the width (in a lateral direction in FIG. 5) of the second region 50b can be set to be smaller than the width of the first region 50a, but is sized to secure at least the stroke of the X-stage 40 in movement.

The X-stage comprises a feed screw 41 extending in a direction of arrow A (X-direction) in the figure, a shaft 42 linked to an end of the feed screw 41, a motor 43 provided outside the vacuum chamber 50 to drive the shaft 42 to rotate, a female screw 45 that mates with the feed screw 41, a stage portion 44 fixed to the female screw 45, and a base 46 fixed to the bottom of the first region 50a and connected to the other end of the feed screw 41. When the motor 43 is driven, the feed screw 41 is rotated at the same position via the shaft 42, and thereby the female screw 45 and the stage portion 44 move in the X-direction. A through hole is made in the stage portion 44, and the rotating stage 1 is inserted into the through hole. The rotating stage 1 is fixed at its flange 80 to and supported by the stage 40, the flange 80 being formed protruding from the periphery of the rotating stage 1. By this means, the rotating stage 1 can move in the X-direction as the X-stage 40 moves. A disc master 200 is mounted on the turn table 5 on the rotating stage, and at the same time that the rotating stage 1 rotates, the X-stage 40 moves in a disc radial direction (a direction of arrow A in FIG. 5), and thereby the positioning control of the electron beam irradiation position is performed.

The rotating stage 1 is mounted such that the lower portion thereof passed through the X-stage 40 is in the second region 50b. Thus, the flexible tubes 16 to 19 and the feeder line 25 attached to the housing bottom of the rotating stage 1 are laid in the second region. The feedthroughs A1 to A5 described above are provided on the side walls of the second region 50b, and between these and the feedthroughs B1 to B5 provided on the housing bottom of the rotating stage, the flexible tubes 16 to 19 and the feeder line 25 are connected to be shaped substantially like a “U”. These pipes and wires hang down in midair in such a way as not to touch a side or bottom on the inside of the vacuum chamber during the movement of the X-stage 40.

Where a vibration-free deck is used to remove vibrations occurring in the apparatus, a through hole is formed in the vibration-free deck 90, and the second region 50b is passed through this through hole, and the bottom of the first region 50a is mounted on the vibration-free deck 90, thereby securing stability.

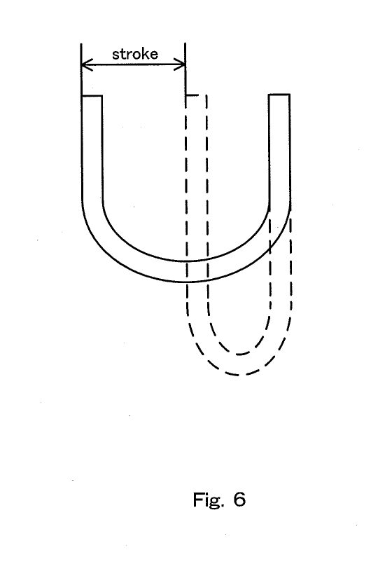

The movement of the flexible tubes 16 to 19 when the X-stage 40 moves in the X-direction will be described with reference to FIG. 6. Each of the flexible tubes hangs down in the middle part to be shaped like a “U” in between the housing bottom of the rotating stage and the vacuum bulkhead 14 inside the second region 50b of the vacuum chamber as described above. In this state, as the X-stage 40 moves in the X-direction, the rotating stage 1 moves in the X-direction. The flexible tube responds to the movement of the X-stage by changing its radius of curvature as shown in FIG. 6. Because each of the flexible tubes hangs down in midair in such a way as not to touch the vacuum bulkhead 14 of the vacuum chamber during the movement of the X-stage, nonlinear disturbance such as friction is eliminated.

As to the flexural rigidity of the flexible tube, complex computation is needed because actually the shape of corrugation needs to be taken into account, but if simply modeled on the assumption that conditions do not change, it can be regarded as the flexural rigidity of a circular pipe. That is, since being proportional to the second moment of area, the flexural rigidity is proportional to the inner diameter cubed for circular pipes of the same wall thickness. In contrast, in the case where multiple pipes of the same inner diameter are arranged in parallel, the second moment of area is proportional to the number of the pipes. Therefore, instead of using a flexible tube of a large inner diameter so that multiple pipes can extend through it as in conventional apparatuses, according to the present embodiment the pipes for air supply and exhaust and for power feeding are individually provided so that each pipe is of a small inner diameter, thereby reducing the flexural rigidity of the pipe. By this means, the movement load on the X-stage can be reduced, achieving highly accurate positioning control.

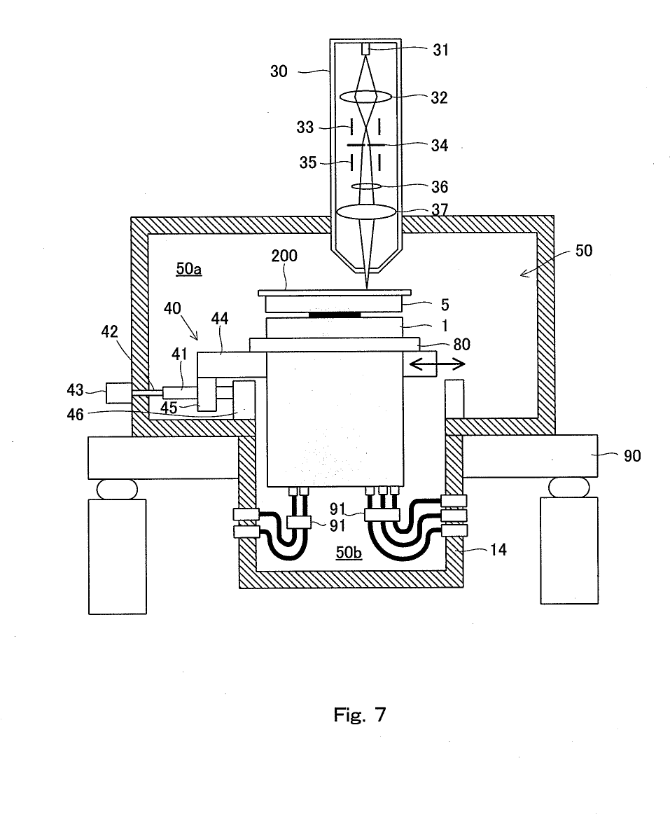

Depending on the lengths and installation intervals of the pipes, the flexible tubes bunging down in the middle part to be shaped like a “U” may be in contact with each other, which causes frictional resistance, resulting in a decrease in the movement accuracy of the X-stage 40. In order to prevent this, it is desirable that clampers 91 to clamp tubes so as to be apart from each other be provided at appropriate places as shown in FIG. 7. Also, a vibration occurring in the U-shaped pipe is expected to be damped by providing this clamper. Although a stainless-made flexible tube usually used as a metallic flexible tube for vacuum, alone is not good at damping, by clamping multiple flexible tubes, vibration energy is dispersed, and thus resonance can be suppressed to be small. Note that covering a metallic flexible tube with a flexible braid (braided wire) or the like is expected to produce the effect of damping vibration. Further, pipes connecting feedthroughs may be, for example, resin-made tubes outgassing little in a vacuum, not being limited to metallic flexible tubes.

Although in the above embodiment, the feedthroughs are provided on the bottom of the housing 13 of the rotating stage 1, not being limited to this, the feedthroughs B1 to B5 may be provided on the lower portion of the side of the rotating stage located in the second region 50b of the vacuum chamber, and between these and the feedthroughs A1 to A5 provided on the vacuum bulkheads 14, the flexible tubes 16 to 19 and the feeder line 25 may be connected hanging down in the middle part to be shaped substantially like a “U”. Also in this case, each of the U-shaped pipes is laid so as not to touch a side or bottom on the inside of the vacuum chamber 50 during the movement of the X-stage 40.

As obvious from the above description, in the stage mechanism and the electron beam lithography apparatus according to the present invention, the vacuum chamber has the trench-like space that spreads underneath the X-stage, and the pipes and wires connected between the rotating stage and the vacuum bulkheads are laid in this space. These pipes and wires are laid so as to deform in response to the movement of the X-stage without touching an inside wall of the vacuum chamber in this space, and hence frictional resistance and vibration during the movement of the X-stage can be greatly reduced. Therefore, the positioning control of a disc master can be executed more highly accurately, and thus minute pits suitable for high density recording can be formed. By providing flexible tubes of a small inner diameter as these pipes respectively individually, the movement load on the X-stage can be further reduced. Further, because the apparatus can be configured without parts such as bellows or guiding tubes used in conventional apparatuses, both low cost and high reliability can be achieved.

1. A stage mechanism which comprises a positioning mechanism including a rotating stage having a turn table, and a linear movement stage to linearly move said rotating stage for positioning; a vacuum chamber housing said positioning mechanism; and at least one flexible pipe to make the outside of said vacuum chamber and the inside of said positioning mechanism communicate, wherein:
said vacuum chamber has a lower space that spreads underneath the underside of said positioning mechanism, and
said pipe leads from a surface of said positioning mechanism through said lower space to an inside wall of said vacuum chamber.

2. A stage mechanism according to claim 1, wherein said pipe hangs down in the middle part without touching an inside wall of said vacuum chamber in said lower space.

3. A stage mechanism according to claim 1, wherein one end of said pipe is coupled to the underside of said positioning mechanism.

4. A stage mechanism according to claim 1, wherein one end of said pipe is coupled to a side surface of said positioning mechanism.

5. A stage mechanism according to claim 1, which has a plurality of said pipes, further comprising a clamper to clamp said pipes so as to be apart from each other.

6. A stage mechanism according to claim 1, wherein:
said linear movement stage has a through hole, and
said rotating stage is inserted into said through hole.

7. An electron beam lithography apparatus which comprises a positioning mechanism including a rotating stage having a turn table, and a linear movement stage to linearly move said rotating stage for positioning; an electron beam irradiating part to irradiate an electron beam onto a disc master mounted on said rotating stage to form record marks; a vacuum chamber housing said positioning mechanism; and at least one flexible pipe to make the outside of said vacuum chamber and the inside of said positioning mechanism communicate, wherein:
said vacuum chamber has a lower space that spreads underneath the underside of said positioning mechanism, and
said pipe leads from a surface of said positioning mechanism through said lower space to an inside wall of said vacuum chamber.
